(19) United States
(12) Patent Application Publication
(10) Pub. No.: US 2009/0178149 A1
(43) Pub. Date: Jul. 9, 2009

(54) Polyamine Sensors and Methods of Using the Same

(76) Inventors: Sylvie Lalonde, Washington, DC (US); Wolf B. Frommer, Washington, DC (US); Loren L. Looger, Washington, DC (US)

Correspondence Address: MORGAN LEWIS & BOCKIUS LLP, 1111 PENNSYLVANIA AVENUE NW, WASHINGTON, DC, 20004, US

(21) Appl. No.: 11/817,478

(22) PCT Filed: Oct. 14, 2005
(86) PCT No.: PCT/US05/36952

§ 371 Date: Mar. 4, 2009

Related U.S. Application Data
(60) Provisional application No. 60/657,702, filed on Mar. 3, 2005.

(51) Int. Cl. A01K 67/027; C12N 15/11; C12N 15/00; C12N 1/21; C12N 1/19; C12N 5/06; C12Q 1/02

(52) U.S. Cl. 800/13; 536/23.7; 435/320.1; 435/252.3; 435/254.2; 435/325; 435/29

(57) ABSTRACT

Polyamine biosensors are disclosed, including putrescine binding biosensors, comprising a polyamine binding domain conjugated to donor and fluorescent moieties that permit detection and measurement of Fluorescence Resonance Energy Transfer upon binding polyamine. Such biosensors are useful for the detection of polyamine concentrations in vivo and in culture.

### CROSS-REFERENCE TO RELATED APPLICATIONS

This application is entitled to priority to U.S. Provisional Patent Application No. 60/657,702, filed Mar. 3, 2005, which is incorporated herein in its entirety. This application is related to provisional application Ser. No. 60/643,576, provisional application Ser. No. 60/658,141, provisional application Ser. No. 60/658,142, provisional application Ser. No. 60/657,702, PCT application [Attorney Docket No. 056100-5053, “Phosphate Biosensors and Methods of Using the Same”], and PCT application [Attorney Docket No. 056100-5054, “Methods of Reducing Repeat-Induced Silencing of Transgene Expression and Improved Fluorescent Biosensors”], which are herein incorporated by reference in their entireties.

### STATEMENT REGARDING FEDERALLY SPONSORED RESEARCH OR DEVELOPMENT

This invention was funded through two grants, including an NIH subcontract from Duke University (Subcontract No. SPSID 126632) and a Human Frontier Science Program grant (Contract No. RGP0041/2004C). Accordingly, the U.S. Government has certain rights to this invention.

### FIELD OF INVENTION

The invention relates generally to the field of polyamine regulation of cell functions such as cell growth and proliferation and, more specifically, to biosensors and methods for measuring and detecting changes in polyamine levels using fluorescence resonance energy transfer (FRET).

### BACKGROUND OF INVENTION

All publications and patent applications herein are incorporated by reference to the same extent as if each individual publication or patent application was specifically and individually indicated to be incorporated by reference.

The publications discussed herein are provided solely for their disclosure prior to the filing date of the present application. Nothing herein is to be construed as an admission that the present invention is not entitled to antedate such publication by virtue of prior invention.

In recent years, a great deal of attention has been focussed on the polyamines, e.g., spermidine, norspermidine, homospermidine, 1,4-diaminobutane (putrescine) and spermine. Polyamines are important for several cell functions. Polyamines affect biological of proteins, stabilize the conformation of nucleic acids, are involved in cell growth and cell proliferation, and they modulate the activity of several ion pumps and channels (Wallace, Fraser and Hughes (2003) A perspective of polyamine metabolism. Biochemical Journal 376, 1-14). In plants, the polyamines when conjugated in the form of hydroxycinnamic acid amides, have been involved in plant defense mechanisms (Walters, (2003) Polyamines and plant diseases. Phytochemistry 64, 97-107).

Polyamines are present in all organisms from prokaryotes to eukaryotes and in eukaryotes they are present virtually in all cell types with a predominance in actively dividing cells. Cell homeostasis is strictly controlled and because of that studying their role in null-mutants (for the different enzymes involved in the biosynthesis) is very difficult. Homeostasis is controlled by uptake, export, synthesis and degradation. Polyamines are synthesized from arginine or ornithine via a decarboxylation step. Consequently, ornithine decarboxylase has been qualified as an oncogene and has been the target for anti-cancer drugs (Wallace et al. 2003).

Studies of polyamines have been largely conducted in the context of cancer probably because of the role they play in proliferative processes. It was shown early on that the polyamine levels in dividing cells, e.g., cancer cells, are much higher than in resting cells. See Janne et al, A. Biochim. Biophys. Acta., Vol. 473, page 241 (1978); Fillingame et al, Proc. Natl. Acad. Sci. U.S.A., Vol. 72, page 4042 (1975); Metcalf et al, J. Am. Chem. Soc., Vol. 100, page 2551 (1978); Flink et al, Nature (London), Vol. 253, page 62 (1975); and Pegg et al, Polyamine Metabolism and Function, Am. J. Cell. Physiol., Vol. 243, pages 212-221 (1982).

Several lines of evidence indicate that polyamines, particularly spermidine, are required for cell proliferation: (i) they are found in greater amounts in growing than in non-growing tissues; (ii) prokaryotic and eukaryotic mutants deficient in polyamine biosynthesis are auxotrophic for polyamines; and (iii) inhibitors specific for polyamine biosynthesis also inhibit cell growth. Despite this evidence, the precise biological role of polyamines in cell proliferation is uncertain. It has been suggested that polyamines, by virtue of their charged nature under physiological conditions and their conformational flexibility, might serve to stabilize macromolecules such as nucleic acids by anion neutralization. See Dkystra et al, Science, Vol. 149, page 48 (1965); Russell et al, Polyamines as Biochemical Markers of Normal and Malignant Growth (Raven, New York, 1978); Hirschfield et al, J. Bacteriol., Vol. 101, page 725 (1970); Hafner et al, J. Biol. Chem., Vol. 254, page 12419 (1979); Cohn et al, J. Bacteriol., Vol. 134, page 208 (1978); Pohjatipelto et al, Nature (London), Vol. 293, page 475 (1981); Mamont et al, Biochem. Biophys. Res. Commun., Vol. 81, page 58 (1978); Bloomfield et al, Polyamines in Biology and Medicine (D. R. Morris and L. J. Morton, eds., Dekker, New York, 1981), pages 183-205; Gosule et al, Nature, Vol. 259, page 333 (1976); Gabbay et al, Ann. N.Y. Acad. Sci., Vol. 171, page 810 (1970); Suwalsky et al, J. Mol. Biol., Vol. 42, page 363 (1969); and Liquori et al, J. Mol. Biol., Vol. 24, page 113 (1968).

Regardless of the reason for increased polyamine levels, the phenomenon can be and has been exploited in chemotherapy. See Sjoerdsma et al, Butterworths Int. Med. Rev.: Clin. Pharmacol. Thera., Vol. 35, page 287 (1984); Israel et al, J. Med. Chem., Vol. 16, page 1 (1973); Morris et al, Polyamines in Biology and Medicine, Dekker, New York, page 223 (1981); and Wang et al, Biochem. Biophys. Res. Commun., Vol. 94, page 85 (1980). Because of the role the natural polyamines play in proliferation, a great deal of effort has been invested in the development of polyamine analogs as anti-proliferatives. Analogues of putrescine, spermidine and spermine have also been tested for their action as anti-cancer drugs with more or less negative results (Seiler, (2003a) Thirty years of polyamine-related approaches to cancer therapy. Retrospect and Prospect. Part 1 Selective enzyme inhibitors. Current Drug Targets 4: 537-564; Seiler, (2003b) Thirty years of polyamine-related approaches to cancer therapy. Retrospect and Prospect. Part 2 Structural analogs and derivatives. Current Drug Targets 4: 565-585).

In addition to cell proliferation, polyamines also play a role in neuronal regeneration. It has been shown that spermine, spermidine and putrescine promote axonal regeneration of lesioned hippocampal neurons (Chu P et al.). Putrescine, spermine and spermidine injected subcutaneously into rats increased immunohistochemically detectable nerve growth factor (Gilad G. et al). Transgenic mice overexpressing ornithine decarboylase, which had high tissue putrescine levels were found on Northern blot analysis to have elevated mRNA levels of brain-derived neuronotrophic factor (BDNF), nerve growth factor (NGF), and neurotrophin-3 (NT-3) in hippocampus (Reeben M. et al).

Despite a number of studies showing the involvement of higher polyamine concentration in proliferative diseases, measuring polyamine concentration in living cells remains challenging. One of the most important tools required to assign functions of cells in vivo would be to visualize polyamine involvement directly. Techniques such as in vivo microdialysis may be used to measure the polyamine concentrations in cells. However, microdialysis is limited in spatial and temporal resolution and the technique itself is destructive to cells.

In vivo measurement of ions and metabolites by using Fluorescence Resonance Energy Transfer (FRET) has been successfully used to measure calcium concentration changes, by fusing CFP, YFP, and a reporter domain consisting of calmodulin and the M13 peptide (Zhang, J., Campbell, R. E., Ting, A. Y., and Tsien, R. Y. (2002a) Creating new fluorescent probes for cell biology. Nat Rev Mol Cell Biol 3, 906-918; Zhang, J., Campbell, R. E., Ting, A. Y., and Tsien, R. Y. (2002b) Creating new fluorescent probes for cell biology. Nature Reviews Molecular Cell Biology 3, 906-918). Binding of calcium to calmodulin causes global structural rearrangement of the chimera resulting in a change in FRET intensity as mediated by the donor and acceptor fluorescent moieties. Recently a number of bacterial periplasmic binding proteins, which undergo a venus flytrap-like closure of two lobes upon substrate binding, have been successfully used as the scaffold of metabolite nanosensors (Fehr, M., Frommer, W. B., and Lalonde, S. (2002) Visualization of maltose uptake in living yeast cells by fluorescent nanosensors. Proc. Natl. Acad. Sci. USA 99, 9846-9851; Fehr, M., Lalonde, S., Lager, I., Wolff, M. W., and Frommer, W. B. (2003) In vivo imaging of the dynamics of glucose uptake in the cytosol of COS-7 cells by fluorescent nanosensors. J. Biol. Chem. 278, 19127-19133; Lager, I., Fehr, M., Frommer, W. B., and Lalonde, S. (2003) Development of a fluorescent nanosensor for ribose. FEBS Lett 553, 85-89). It would be useful if a FRET biosensor could be developed for measuring polyamine levels.

### SUMMARY OF INVENTION

The present invention provides polyamine biosensors for detecting and measuring changes in polyamine concentrations. In particular, the invention provides an isolated nucleic acid encoding a polyamine binding fluorescent indicator comprising a polyamine binding protein moiety from a bacterium, such as Escherichia coli or Agrobacterium tumefaciens, wherein the polyamine binding protein moiety is covalently coupled to a donor fluorescent protein moiety and an acceptor fluorescent protein moiety, and wherein fluorescence resonance energy transfer (FRET) between the donor moiety and the acceptor moiety is altered when the donor moiety is excited and polyamine binds to the polyamine binding protein moiety. Vectors, including expression vectors, and host cells comprising the inventive nucleic acids are also provided, as well as biosensor proteins encoded by the nucleic acids. Such nucleic acids, vectors, host cells and proteins may be used in methods of detecting changes in polyamine levels and particularly polyamine levels in proliferating and cancerous cells, and in methods of identifying compounds that modulate polyamine activity.

### BRIEF DESCRIPTION OF THE DRAWINGS

FIG. 1 shows a titration curve of FLIP-AF1 against various putrescine concentrations. The binding affinity is 0.211 μM. Examples are of two independent clones.

### DETAILED DESCRIPTION OF INVENTION

The following description includes information that may be useful in understanding the present invention. It is not an admission that any of the information provided herein is prior art or relevant to the presently claimed inventions, or that any publication specifically or implicitly referenced is prior art.

Other objects, advantages and features of the present invention become apparent to one skilled in the art upon reviewing the specification and the drawings provided herein. Thus, further objects and advantages of the present invention will be clear from the description that follows.

### Biosensors

The present invention provides polyamine biosensors for detecting and measuring changes in polyamine concentrations using Fluorescence Resonance Energy Transfer (FRET). In particular, the invention provides polyamine binding fluorescent indicators, particularly indicators comprising a polyamine binding protein moiety from a bacterium, and more specifically a putrescine (1,4-diaminobutane) binding moiety from the Agrobacterium tumefaciens. Additional polyamine biosensors for other polyamines such as spermidine, norspermidine, homospermidine and spermine, as well as polyamine binding proteins from other bacterial species, may also be prepared using the constructs and methods provided herein.

For instance, PotD and PotF are two known polyamine binding proteins from E. coli differing in their specificity, i.e., PotD is spermidine-preferential and PotF is putrescine-preferential (see Igarashi and Kashiwagi, 1999, Biochem. J. 344: 633-42, which is herein incorporated by reference in its entirety). We used a homology database search to identify a putative homolog from Agrobacterium (AF1), and have now demonstrated that this homolog which has not previously been characterized as a polyamine binding protein can be turned into a polyamine sensor. This is proof of concept that other proteins which are homologous to E. coli PotD and PotF, and/or Agrobacterium AF1, can also be used to construct polyamine sensors according to the present invention. It is further possible according to the methods provided herein to perform mutagenesis of the binding pocket with or without computational design to modify the binding specificity, for instance to change the polyamine preference of a given binding moiety.

Thus, the invention provides isolated nucleic acids encoding polyamine binding fluorescent indicators. One embodiment, among others, is an isolated nucleic acid which encodes a polyamine binding fluorescent indicator, the indicator comprising: a polyamine binding protein moiety, a donor fluorescent protein moiety covalently coupled to the polyamine binding protein moiety, and an acceptor fluorescent protein moiety covalently coupled to the polyamine binding protein moiety, wherein FRET between the donor moiety and the acceptor moiety is altered when the donor moiety is excited and polyamine binds to the polyamine binding protein moiety.

“Covalently coupled” means that the donor and acceptor fluorescent moieties may be conjugated to the polyamine binding protein moiety via a chemical linkage, for instance to a selected amino acid in said polyamine binding protein moiety. Covalently coupled also means that the donor and acceptor moieties may be genetically fused to the polyamine binding protein moiety such that the polyamine binding protein moiety is expressed as a fusion protein comprising the donor and acceptor moieties.

A preferred polyamine binding protein moiety, among others, is a putrescine binding protein moiety from Agrobacterium tumefaciens AF1 protein. The DNA sequence of AF1 (SEQ ID No. 1) and its protein sequence (AF1, protein accession no NP_{—}531310, SEQ ID No. 2) are known in the art. Any portion of the AF1 DNA sequence which encodes a putrescine binding region may be used in the nucleic acids of the present invention. Putrescine binding portions of AF1 or any of its homologues, including polyamine binding portions of E. coli PotD and PotF, may be cloned into the vectors described herein and screened for activity according to the disclosed assays.

Naturally occurring species variants of AF1, PotD and PotF may also be used. For instance, a database search of National Cancer Institutes' protein database reveals that homologues for PotD and PotF have been isolated from a variety of organisms and bacterial species, any of which may be used to construct a polyamine sensor according to the methods of the present invention. For instance, such organisms include, but are not limited to, plants such as Arabidopsis, and bacterial species of Erwinia, Haemophilus, Streptococcus, Neisseria, Gluconobacter, Lactobacillus, Vibrio, Staphylococcus, Salmonella, Shigella, Yersinia, Burkholderia, Bordetella, to name a few.

In addition, artificially engineered variants of the polyamine binding moieties described herein may also be used, i.e., that comprise site-specific mutations, deletions or insertions while maintaining measurable polyamine binding function. Variant nucleic acid sequences suitable for use in the nucleic acid constructs of the present invention will preferably have at least 70, 75, 80, 85, 90, 95, or 99% similarity or identity to the native bacterial polyamine binding gene sequence, for instance AF1. Suitable variant nucleic acid sequences may also hybridize to the native gene, i.e., AF1, under highly stringent hybridization conditions. High stringency conditions are known in the art; see for example Maniatis et al., Molecular Cloning: A Laboratory Manual, 2d Edition, 1989, and Short Protocols in Molecular Biology, ed. Ausubel, et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993), which is herein incorporated by reference. Generally, stringent conditions are selected to be about 5-10° C. lower than the thermal melting point (Tm) for the specific sequence at a defined ionic strength pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 11.0M sodium ion, typically about 0.01 to 1.0M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide.

Preferred artificial variants of the present invention may exhibit decreased affinity for polyamine, in order to expand the range of concentration that can be measured by AF1-based and other polyamine nanosensors. Additional artificial variants showing decreased or increased binding affinity for polyamine may be constructed by random or site-directed mutagenesis and other known mutagenesis techniques, and cloned into the vectors described herein and screened for activity according to the disclosed assays.

The isolated nucleic acids of the invention may incorporate any suitable donor and acceptor fluorescent protein moieties that are capable in combination of serving as donor and acceptor moieties in FRET. Preferred donor and acceptor moieties are selected from the group consisting of GFP (green fluorescent protein), CFP (cyan fluorescent protein), BFP (blue fluorescent protein), YFP (yellow fluorescent protein), and enhanced variants thereof, with a particularly preferred embodiment provided by the donor/acceptor pair CFP/YFP Venus, a variant of YFP with improved pH tolerance and maturation time (Nagai, T., Ibata, K., Park, E. S., Kubota, M., Mikoshiba, K., and Miyawaki, A. (2002) A variant of yellow fluorescent protein with fast and efficient maturation for cell-biological applications. Nat. Biotechnol. 20, 87-90). An alternative is the MiCy/mKO pair with higher pH stability and a larger spectral separation (Karasawa S, Araki T, Nagai T, Mizuno H, Miyawaki A. Cyan-emitting and orange-emitting fluorescent proteins as a donor/acceptor pair for fluorescence resonance energy transfer. Biochem J. 2004 381:307-12). Criteria to consider when selecting donor and acceptor fluorescent moieties is known in the art, for instance as disclosed in U.S. Pat. No. 6,197,928, which is herein incorporated by reference in its entirety.

Also suitable as either a donor or acceptor is native DsRed from a Discosoma species, an ortholog of DsRed from another genus, or a variant of a native DsRed with optimized properties (e.g. a K83M variant or DsRed2 (available from Clontech)). As used herein, the term “variant” is intended to refer to polypeptides with at least about 70%, more preferably at least 75% identity, including at least 80%, 90%, 95% or greater identity to native fluorescent molecules. Many such variants are known in the art, or can be readily prepared by random or directed mutagenesis of a native fluorescent molecules (see, for example, Fradkov et al., FEBS Lett. 479:127-130 (2000)).

When the fluorophores of the biosensor contain stretches of similar or related sequence(s), the present inventors have recently discovered that gene silencing may adversely affect expression of the biosensor in certain cells and particularly whole organisms. In such instances, it is possible to modify the fluorophore coding sequences at one or more degenerate or wobble positions of the codons of each fluorophore, such that the nucleic acid sequences of the fluorophores are modified but not the encoded amino acid sequences. Alternative, one or more conservative substitutions that do not adversely affect the function of the fluorophores may also be incorporated. See PCT application [Attorney Docket No. 056100-5054, “Methods of Reducing Repeat-Induced Silencing of Transgene Expression and Improved Fluorescent Biosensors], which is herein incorporated by reference in its entirety.

The invention further provides vectors containing isolated nucleic acid molecules encoding polyamine biosensor polypeptides. Exemplary vectors include vectors derived from a virus, such as a bacteriophage, a baculovirus or a retrovirus, and vectors derived from bacteria or a combination of bacterial sequences and sequences from other organisms, such as a cosmid or a plasmid. Such vectors include expression vectors containing expression control sequences operatively linked to the nucleic acid sequence coding for the polyamine biosensor. Vectors may be adapted for function in a prokaryotic cell, such as E. coli or other bacteria, or a eukaryotic cell, including yeast and animal cells. For instance, the vectors of the invention will generally contain elements such as an origin of replication compatible with the intended host cells, one or more selectable markers compatible with the intended host cells and one or more multiple cloning sites. The choice of particular elements to include in a vector will depend on factors such as the intended host cells, the insert size, whether regulated expression of the inserted sequence is desired, i.e., for instance through the use of an inducible or regulatable promoter, the desired copy number of the vector, the desired selection system, and the like. The factors involved in ensuring compatibility between a host cell and a vector for different applications are well known in the art.

Preferred vectors for use in the present invention will permit cloning of the polyamine binding domain or receptor between nucleic acids encoding donor and acceptor fluorescent molecules, resulting in expression of a chimeric or fusion protein comprising the polyamine binding domain covalently coupled to donor and acceptor fluorescent molecules. Exemplary vectors include the bacterial pRSET-FLIP derivatives disclosed in Fehr et al. (2002) (Visualization of maltose uptake in living yeast cells by fluorescent nanosensors. Proc. Natl. Acad. Sci. USA 99, 9846-9851), which is herein incorporated by reference in its entirety. Methods of cloning nucleic acids into vectors in the correct frame so as to express a fusion protein are well known in the art.

The chimeric nucleic acids of the present invention are preferably constructed such that the donor and acceptor fluorescent moiety coding sequences are fused to separate termini of the polyamine binding domain in a manner such that changes in FRET between donor and acceptor may be detected upon polyamine binding. Fluorescent domains can optionally be separated from the polyamine binding domain by one or more flexible linker sequences. Such linker moieties are preferably between about 1 and 50 amino acid residues in length, and more preferably between about 1 and 30 amino acid residues. Linker moieties and their applications are well known in the art and described, for example, in U.S. Pat. Nos. 5,998,204 and 5,981,200, and Newton et al., Biochemistry 35:545-553 (1996). Alternatively, shortened versions of any of the fluorophores described herein may be used.

It will also be possible depending on the nature and size of the polyamine binding domain to insert one or both of the fluorescent molecule coding sequences within the open reading frame of the polyamine binding protein such that the fluorescent moieties are expressed and displayed from a location within the biosensor rather than at the termini. Such sensors are generally described in U.S. Application Ser. No. 60/658,141, which is herein incorporated by reference in its entirety. It will also be possible to insert a polyamine binding sequence, such as a sequence encoding AF1 or other polyamine binding domain, into a single fluorophore coding sequence, i.e. a sequence encoding a GFP, YFP, CFP, BFP, etc., rather than between tandem molecules. According to the disclosures of U.S. Pat. No. 6,469,154 and U.S. Pat. No. 6,783,958, each of which is incorporated herein by reference in their entirety, such sensors respond by producing detectable changes within the protein that influence the activity of the fluorophore.

The invention also includes host cells transfected with a vector or an expression vector of the invention, including prokaryotic cells, such as E. coli or other bacteria, or eukaryotic cells, such as yeast cells or animal cells. In another aspect, the invention features a transgenic non-human animal having a phenotype characterized by expression of the nucleic acid sequence coding for the expression of the polyamine biosensor. The phenotype is conferred by a transgene contained in the somatic and germ cells of the animal, which may be produced by (a) introducing a transgene into a zygote of an animal, the transgene comprising a DNA construct encoding the polyamine biosensor; (b) transplanting the zygote into a pseudopregnant animal; (c) allowing the zygote to develop to term; and (d) identifying at least one transgenic offspring containing the transgene. The step of introducing of the transgene into the embryo can be by introducing an embryonic stem cell containing the transgene into the embryo, or infecting the embryo with a retrovirus containing the transgene. Transgenic animals of the invention include transgenic C. elegans and transgenic mice and other animals.

The present invention also encompasses isolated polyamine biosensor molecules having the properties described herein, particularly AF1-based polyamine binding fluorescent indicators. Such polypeptides may be recombinantly expressed using the nucleic acid constructs described herein, or produced by chemically coupling some or all of the component domains. The expressed polypeptides can optionally be produced in and/or isolated from a transcription-translation system or from a recombinant cell, by biochemical and/or immunological purification methods known in the art. The polypeptides of the invention can be introduced into a lipid bilayer, such as a cellular membrane extract, or an artificial lipid bilayer (e.g. a liposome vesicle) or nanoparticle.

### Methods of Detecting Levels of Polyamines

The nucleic acids and proteins of the present invention are useful for detecting and measuring changes in the levels of polyamines in the organs of an animal. In one embodiment, the invention comprises a method of detecting changes in the level of polyamine in a sample of cells, comprising (a) providing a cell expressing a nucleic acid encoding a polyamine binding biosensor as described herein and a sample of cells; and (b) detecting a change in FRET between a donor fluorescent protein moiety and an acceptor fluorescent protein moiety, each covalently attached to the polyamine binding domain, wherein a change in FRET between said donor moiety and said acceptor moiety indicates a change in the level of polyamine in the sample of cells.

FRET may be measured using a variety of techniques known in the art. For instance, the step of determining FRET may comprise measuring light emitted from the acceptor fluorescent protein moiety. Alternatively, the step of determining FRET may comprise measuring light emitted from the donor fluorescent protein moiety, measuring light emitted from the acceptor fluorescent protein moiety, and calculating a ratio of the light emitted from the donor fluorescent protein moiety and the light emitted from the acceptor fluorescent protein moiety. The step of determining FRET may also comprise measuring the excited state lifetime of the donor moiety or anisotropy changes (Squire A, Verveer P J, Rocks O, Bastiaens P I. J Struct Biol. 2004 July; 147(1):62-9. Red-edge anisotropy microscopy enables dynamic imaging of homo-FRET between green fluorescent proteins in cells.). Such methods are known in the art and described generally in U.S. Pat. No. 6,197,928, which is herein incorporated by reference in its entirety.

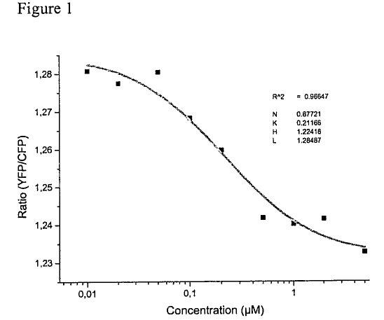

The amount of polyamine or other polyamine in a sample of cells can be determined by determining the degree of FRET. First the sensor must be introduced into the sample. Changes in polyamine concentration can be determined by monitoring FRET at a first and second time after contact between the sample and the fluorescent indicator and determining the difference in the degree of FRET. The amount of polyamine in the sample can be quantified for example by using a calibration curve established by titration (FIG. 1).

The cell sample to be analyzed by the methods of the invention may be contained in vivo, for instance in the measurement of ligand transport on the surface of cells, or in vitro, wherein ligand efflux may be measured in cell culture. Alternatively, a fluid extract from cells or tissues may be used as a sample from which ligands are detected or measured.

Methods for detecting polyamine levels as disclosed herein may be used to screen and identify compounds that may be used to modulate polyamine concentrations and particularly compounds useful for modulating polyamine activity. In one embodiment, among others, the invention comprises a method of identifying a compound that modulates polyamine activity comprising (a) contacting a mixture comprising a cell expressing a polyamine biosensor as disclosed herein and a sample of cells with one or more test compounds, and (b) determining FRET between said donor fluorescent domain and said acceptor fluorescent domain following said contacting, wherein increased or decreased FRET following said contacting indicates that said test compound is a compound that modulates polyamine activity.

The term “modulate” in this embodiment means that such compounds may increase or decrease polyamine activity. Compounds that increase polyamine levels are targets for therapeutic intervention and treatment of disorders associated with polyamine activity, as described above. Compounds that decrease polyamine levels may be developed into therapeutic products for the treatment of disorders associated with polyamine activity, such as cancer, autoimmune diseases and other proliferative disorders. Cancers to be treated by such compounds include any type of cancer, including but not limited to breast, brain, skin, blood, pancreatic, ovarian, prostate, kidney, bone, etc.

The targeting of the sensor to the outer leaflet of the plasma membrane is only one embodiment of the potential applications. It demonstrates that the nanosensor can be targeted to a specific compartment. Alternatively, other targeting sequences may be used to express the sensors in other compartments such as vesicles, ER, vacuole, etc.

### Additional Utilities

The biosensors of the present invention can also be expressed on the surface of animal cells to determine the function of neurons. For example, in C. elegans, many of the neurons present have not been assigned a specific function. Expression of the biosensors on the surface permits visualization of neuron activity in living worms in response to stimuli, permitting assignment of function and analysis of neuronal networks. Similarly, the introduction of multiphoton probes into the brain of living mice or rats permits imaging these processes. Finally, expression in specific neurons or glia will allow the study of phenomena such as stroke or Alzheimers Disease and the effect of such disorders on polyamine levels inside neuronal cells or on their surface. Moreover, the effect of medication on localized brain areas or neuronal networks can be studied in vivo.

Finally, it is possible to use the sensors as tools to modify polyamine activity, by introducing them as artificial polyamine scavengers, for instance presented on membrane or artificial lipid complexes or expressed in targeted cells, and thus to manipulate cell proliferation or homeostasis.

The following examples are provided to describe and illustrate the present invention. As such, they should not be construed to limit the scope of the invention. Those in the art will well appreciate that many other embodiments also fall within the scope of the invention, as it is described hereinabove and in the claims.

### EXAMPLES

### Example 1

### In Vitro Characterization of FLIP-AF1 Nanosensors

A DNA fragment encoding the mature AF1 protein was fused to CFP and the YFP Venus sequence at the N- and C-termini, respectively. Emission spectra and substrate titration curves were obtained by using monochromator microplate reader Safire (Tecan, Austria). Excitation filter was 433/−12 nm, emission filters for CFP and YFP emission were 485/−12, 528 nm/12 nm, respectively. All analyses were done in 20 mM sodium phosphate buffer, pH 7.0.

To quantify the intensity of CFP and CFP emission, the fluorescence intensity in the two channels in the periphery of the cell was integrated on a pixel-by-pixel basis, and the YFP/CFP ratio was calculated. The FLIP-AF1 biosensor (see SEQ ID Nos. 3 and 4) was titrated against various putrescine concentrations. FIG. 1 shows that addition of putrescine resulted in an increase in CFP emission and a decrease in YFP emission (a net effect of decrease in the YFP/CFP ratio). The binding affinity of putrescine was determined to be 0.211 μM.

All publications, patents and patent applications discussed herein are incorporated herein by reference. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed:

1. An isolated nucleic acid which encodes a polyamine binding fluorescent indicator, the indicator comprising:
a polyamine binding protein moiety from a microorganism;
a donor fluorescent protein moiety covalently coupled to the polyamine binding protein moiety; and
an acceptor fluorescent protein moiety covalently coupled to the polyamine binding protein moiety;
wherein fluorescence resonance energy transfer (FRET) between the donor moiety and the acceptor moiety is altered when the donor moiety is excited and polyamine binds to the polyamine binding protein moiety.

2. The isolated nucleic acid of claim 1, wherein the microorganism is a bacterium.

3. The isolated nucleic acid of claim 2, wherein the bacterium is selected from the group consisting of Escherichia coli and Agrobacterium tumefaciens.

4. The isolated nucleic acid of claim 1, wherein said polyamine binding moiety is a putrescine binding protein.

5. The isolated nucleic acid of claim 1, wherein said polyamine binding moiety is a spermidine binding protein.

6. The isolated nucleic acid of claim 1, wherein said polyamine binding moiety is a spermine binding protein.

7. The isolated nucleic acid of claim 1, wherein said polyamine binding protein moiety comprises the sequence of SEQ ID No. 1.

8. The isolated nucleic acid of claim 1, wherein said donor fluorescent protein moiety is selected from the group consisting of a GFP, a CFP, a BFP, a YFP, a dsRED, CoralHue Midoriishi-Cyan (MiCy) and monomeric CoralHue Kusabira-Orange (mKO).

9. The isolated nucleic acid of claim 1, wherein said acceptor fluorescent protein moiety is selected from the group consisting of a GFP, a CFP, a BFP, a YFP, a dsRED, CoralHue Midoriishi-Cyan (MiCy) and monomeric CoralHue Kusabira-Orange (KO).

10. The isolated nucleic acid of claim 1, wherein said donor fluorescent protein moiety is a CFP and said acceptor fluorescent protein moiety is YFP Venus.

11. The isolated nucleic acid of claim 1, further comprising at least one linker moiety.

12. A cell expressing the nucleic acid of claim 1.

13. An expression vector comprising the nucleic acid of claim 1.

14. A cell comprising the vector of claim 13.

15. The expression vector of claim 13 adapted for function in a prokaryotic cell.

16. The expression vector of claim 13 adapted for function in a eukaryotic cell.

17. The cell of claim 12, wherein the cell is a prokaryote.

18. The cell of claim 17, wherein the cell is Agrobacterium tumefaciens.

19. The cell of claim 12, wherein the cell is a eukaryotic cell.

20. The cell of claim 19, wherein the cell is a yeast cell.

21. The cell of claim 19, wherein the cell is an animal cell.

22. A transgenic animal expressing the nucleic acid of claim 1.

23. The transgenic animal of claim 22, wherein said transgenic animal is C. elegans.

24. The isolated nucleic acid of claim 1, further comprising one or more nucleic acid substitutions that lower the affinity of the polyamine binding protein moiety to polyamine.

25. A polyamine binding fluorescent indicator encoded by the nucleic acid of claim 1.

26. A method of detecting changes in the level of polyamines in a sample of cells, comprising:
(a) providing a cell expressing the nucleic acid of claim 1; and
(b) detecting a change in FRET between said donor fluorescent protein moiety and said acceptor fluorescent protein moiety,
wherein a change in FRET between said donor moiety and said acceptor moiety indicates a change in the level of extracellular polyamine in a sample of neurons.

27. The method of claim 26, wherein the step of determining FRET comprises measuring light emitted from the acceptor fluorescent protein moiety.

28. The method of claim 26, wherein determining FRET comprises measuring light emitted from the donor fluorescent protein moiety, measuring light emitted from the acceptor fluorescent protein moiety, and calculating a ratio of the light emitted from the donor fluorescent protein moiety and the light emitted from the acceptor fluorescent protein moiety.

29. The method of claim 26, wherein the step of determining FRET comprises measuring the excited state lifetime of the donor moiety.

30. The method of claim 26, wherein said sample of cells is contained in vivo.

31. The method of claim 26, wherein said sample of cells is contained in vitro.

32. The method of claim 26, wherein said change in the level of polyamines is associated with cell growth, cell proliferation, ion pump activity, ion channel activity, and one or more plant defense mechanisms.

33. The method of claim 26, wherein said change in the level of polyamines is associated with cancer.

34. A method of identifying a compound that modulates the activity of a polyamine in a cell, comprising:
(a) contacting a cell expressing the nucleic acid of claim 1 with one or more test compounds; and
(b) determining FRET between said donor fluorescent domain and said acceptor fluorescent domain following said contacting,
wherein increased or decreased FRET following said contacting indicates that said test compound is a compound that modulates polyamine activity.

35. The method of claim 34, wherein said compound is a putrescine, spermidine, or spermine analog.

36. The nucleic acid of claim 1, wherein said donor and acceptor fluorescent moieties are genetically fused to said polyamine binding protein moiety.

37. The nucleic acid of claim 36, wherein said donor and acceptor fluorescent moieties are genetically fused to the termini of said polyamine binding moiety.

38. The nucleic acid of claim 36, wherein one or both of said donor and acceptor fluorescent moieties are fused to an internal position of said polyamine binding moiety.


SEQUENCE LISTING (4 sequences)

SEQ ID NO: 1 - DNA, 1098 bp, Agrobacterium tumefaciens str. C58
atgaaaaaccgttctctccgcctgaccttggctctcaccaccgcgctcgttgcagcaggctcggcaatggcccaggagaaggtcgtccacgtctataactggtcggactatatcgatcccgtcatccttgaagattttaccaaggaaaccgggataaaggtggtctacgacgtgtatgacggcaacgaagtgctggagaccaagcttttggccggcagctcgggttatgacgtcgtggcgccgacatcgcctttcctggcgcggcagatcaaggccggtgtctaccagaagctggacaagtccaagctgccgaacctcaagaatgcgtggccggacattaccgaacgcctggcgaaatatgaccccggcaacgaatatgccgtcaactacatgtggggcaccaccggcatcggctacaatgtcgccaaggtgaaggccgcgctcggcgatgtgcctgttgatagctgggacgtgttgttcaagccggaaaatgccgagaagctgaaatcctgtggtatcaacattctcgacgcgtcggacgagactttcgcgattgcgatgaactatctcggcaagaatcccgacagtaaggaaacggccgacctggaagcgggcggcgaggtctattcgaagattcgcccctatgtgaagaccttcaattcatcgccctatatcaatgatctcgccaatggcgacacttgcatctccatcggctggtccggcgatattcttcaggcaaaaacgcgcgccgaggaagccaagaacggcgttgaggtgaattatgtcatccccaaggaaggcacctatatctggatggatagctttgccattccggctgatgccaagaatgtcgatgaggcccatgccttcatcaactacatgatgaagcctgaagtcgccgccaaggcttccgactacgtgcaatacgccaacggcaaccttccctcgcaggcgctgatggatccgtcggtcgcgaagaacccgggggtctatcccgatccggaaacgatgaagaagctcttcaccatttcgccctacggaccgaaggagcagcgtgtgttgaaccgcgtctggacgcagataaagaccggcacctga

SEQ ID NO: 2 - protein, 365 aa, Agrobacterium tumefaciens str. C58
MKNRSLRLTLALTTALVAAGSAMAQEKVVHVYNWSDYIDPVILEDFTKETGIKVVYDVYDGNEVLETKLLAGSSGYDVVAPTSPFLARQIKAGVYQKLDKSKLPNLKNAWPDITERLAKYDPGNEYAVNYMWGTTGIGYNVAKVKAALGDVPVDSWDVLFKPENAEKLKSCGINILDASDETFAIAMNYLGKNPDSKETADLEAGGEVYSKIRPYVKTFNSSPYINDLANGDTCISIGWSGDILQAKTRAEEAKNGVEVNYVIPKEGTYIWMDSFAIPADAKNVDEAHAFINYMMKPEVAAKASDYVQYANGNLPSQALMDPSVAKNPGVYPDPETMKKLFTISPYGPKEQRVLNRVWTQIKTGT

SEQ ID NO: 3 - DNA, 2469 bp, Artificial Sequence
  FLIP-AF1 fusion sequence derived from Agrobacterium tumifaciens AF1 sequence, cyan fluorescent protein of unknown origin, and yellow fluorescent protein of unknown origin
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggtggtaccaaggtcgtccacgtctataactggtcggactatatcgatcccgtcatccttgaagattttaccaaggaaaccgggataaaggtggtctacgacgtgtatgacggcaacgaagtgctggagaccaagcttttggccggcagctcgggttatgacgtcgtggcgccgacatcgcctttcctggcgcggcagatcaaggccggtgtctaccagaagctggacaagtccaagctgccgaacctcaagaatgcgtggccggacattaccgaacgcctggcgaaatatgaccccggcaacgaatatgccgtcaactacatgtggggcaccaccggcatcggctacaatgtcgccaaggtgaaggccgcgctcggcgatgtgcctgttgatagctgggacgtgttgttcaagccggaaaatgccgagaagctgaaatcctgtggtatcaacattctcgacgcgtcggacgagactttcgcgattgcgatgaactatctcggcaagaatcccgacagtaaggaaacggccgacctggaagcgggcggcgaggtctattcgaagattcgcccctatgtgaagaccttcaattcatcgccctatatcaatgatctcgccaatggcgacacttgcatctccatcggctggtccggcgatattcttcaggcaaaaacgcgcgccgaggaagccaagaacggcgttgaggtgaattatgtcatccccaaggaaggcacctatatctggatggatagctttgccattccggctgatgccaagaatgtcgatgaggcccatgccttcatcaactacatgatgaagcctgaagtcgccgccaaggcttccgactacgtgcaatacgccaacggcaaccttccctcgcaggcgctgatggatccgtcggtcgcgaagaacccgggggtctatcccgatccggaaacgatgaagaagctcttcaccatttcgccctacggaccgaaggagcagcgtgtgttgaaccgcgtctggacgcagataaagaccggtaccggtggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 4 - protein, 822 aa, Artificial Sequence
  FLIP-AF1 fusion sequence derived from Agrobacterium tumifaciens AF1 sequence, cyan fluorescent protein of unknown origin, and yellow fluorescent protein of unknown origin
MVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELYKGGTKVVHVYNWSDYIDPVILEDFTKETGIKVVYDVYDGNEVLETKLLAGSSGYDVVAPTSPFLARQIKAGVYQKLDKSKLPNLKNAWPDITERLAKYDPGNEYAVNYMWGTTGIGYNVAKVKAALGDVPVDSWDVLFKPENAEKLKSCGINILDASDETFAIAMNYLGKNPDSKETADLEAGGEVYSKIRPYVKTFNSSPYINDLANGDTCISIGWSGDILQAKTRAEEAKNGVEVNYVIPKEGTYIWMDSFAIPADAKNVDEAHAFINYMMKPEVAAKASDYVQYANGNLPSQALMDPSVAKNPGVYPDPETMKKLFTISPYGPKEQRVLNRVWTQIKTGTGGMVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK